(19) United States
(12) Patent Application Publication
(10) Pub. No.: US 2012/0264109 A1
(43) Pub. Date: Oct. 18, 2012

(54) LYMPHOCYTE ANALYSIS FOR MONITORING THE PROGRESSION OF IMMUNODEFICIENCY VIRUS

(75) Inventors: Chiranjit DEKA, Morrisville, NC (US); Jui Ming LIN, Falmouth, ME (US); Mary GOYETTE, Somersworth, NH (US)

(73) Assignee: IDEXX LABORATORIES, INC., Westbrook, ME (US)

(21) Appl. No.: 13/432,542

(22) Filed: Mar. 28, 2012

Related U.S. Application Data
(60) Provisional application No. 61/474,387, filed on Apr. 12, 2011.

(51) Int. Cl. G01N 33/566

(52) U.S. Cl. 435/5

(57) ABSTRACT

The present disclosure describes a method of monitoring disease progression in a mammal positive for immunodeficiency virus which includes collecting blood cells from a mammal to obtain a first blood sample adding antibodies such as CD4 and CD8 to the first blood sample scanning the blood sample to produce a first multivariate dot plot which may be used to quantify at least CD4⁺ and CD8⁺ blood cell populations to produce a first ratio. The first multivariate dot plot may also be used to quantify a CD8αβ^{low} subpopulation which may be used to calculate a second ratio. A third ratio is calculated of the second ratio to the first ratio and the result plotted on a graph as a first point. This process may be repeated to produce a second point for evaluating an extent of disease progression.

### TECHNICAL FIELD

The present disclosure relates to methods of assessing immunological health of a mammal infected with immunodeficiency virus, such as Feline Immunodeficiency Virus (FIV) or Human Immunodeficiency Virus (HIV). More specifically, the disclosure relates to the use of cellular analysis to facilitate diagnosis and monitoring of the immunodeficiency virus in a mammal.

### BACKGROUND

Enumeration of cluster of differentiation 4 positive (CD4⁺) T-cells is important in the diagnosis and monitoring of HIV in humans. Measuring CD4⁺ lymphocytes in human whole blood samples has been described in the literature. It has been demonstrated that as the virus progresses, the number of CD4⁺ T-cells decrease.

In contrast to the decrease in CD4⁺ T-cells, cluster of differentiation 8 positive (CD8⁺) T-cells may increase in number as the immunodeficiency virus progresses. A common method of identifying and monitoring HIV infection may include monitoring the ratio of CD4³⁰:CD8⁺ T-cells. However, this ratio may not reflect disease progress until months or, in cases, years following infection.

The desire for a method of detecting infection earlier, as well as a desire to understand the reason for the increase in CD8⁺ T-cells during infection, has led to examination of CD8⁺ T-cells. CD8 forms a dimer from two primary isoforms of CD8, alpha (α) and beta (β). The dimer formed by CD8 may be a heterodinier, formed from both the α and the β isoforms, or a homodimer formed from two α isoforms. These isoforms allow for segregation of the CD8⁺ T-cells into subpopulations for further analysis.

Subpopulations of CD8⁺ T-cells include those that express the αβ-complex in high numbers and fluoresce at a higher intensity (CD8αβ^{high}), CD8⁺ T-cells that express the αβ-complex in low numbers fluoresce at a lower intensity (CD8αβ^{low}). These subpopuiations may be separated by a flow cytometer based on their level of fluorescence using specific antibodies that preferentially recognize only the α chain, only the β chain, or the αβ complexes on the CD8⁺ T-cells.

FIV is a lentivirus that infects cats in a manner somewhat similar to HIV infection of humans. Enumeration of CD8⁺ lymphocytes and CD4⁺ lymphocytes as well as measurement of CD4⁺/CD8⁺ in feline blood samples has been described in the literature. As with HIV, the absolute count of CD4⁺ T-cells and the CD4⁺/CD8⁺ ratio in cats decrease as the FIV infection progresses.

Methods of determining cell populations, such as the level of CD4⁺ and CD8⁺, typically involve evaluation of fluorescent labeled leukocytes using a flow cytometer. The CD4⁺ T-cells and the CD8⁺ T-cells may be labeled with different fluorescent conjugated antibodies. The fluorescent antibodies bind to either the CD4⁺ or CD8⁺ receptor site and generate a fluorescent signal. Antibodies specific to the α and β chains or the αβ complex of the CD8⁺ are used to detect cells exhibiting these in different analyzer or separate quantities. The total lymphocyte count per μL of blood is measured in a hematology flow cytometer calibrated to measure absolute lymphocyte counts in whole blood.

The measurement of CD4⁺ T-cells presents certain challenges. For example, low total lymphocyte levels in FIV negative felines may be caused by clinical reasons other than FIV, resulting in false positive results. Additionally, the frequency and manner in which blood is drawn from a cat may also influence the total lymphocyte count. Even FIV negative (FIV⁻) cats, whose CD4⁺ percent is within a normal range, may occasionally exhibit an absolute CD4⁺ count lower than a FIV positive (FIV⁺) cat. Such anomalies have often hindered the interpretation of CD4³⁰ results from FIV⁺ cats.

Additional problems arise in cats that have a CD4⁺:CD8⁺ ratio close to i. FIV⁻ cats generally have a CD4⁺:CD8⁺ ratio greater than 1 whereas the CD4⁺:CD8⁺ ratio of FIV⁺ cats tends to be lower (<1). However, in cats with a CD4⁺:CD8⁺ ratio close to 1, interpretation of the results again becomes difficult.

Several subpopulations of CD8⁺, including CD8αα, CD8α⁺β⁻; CD8αβ^{high} and CD8αβ^{low}, have been identified in both HIV and FIV infected mammals. The relative concentration of these subpopulations within the total lymphocyte population and within the CD8⁺ population vary depending on the stage, duration, and host immune response to infection with the virus. Specific subpopulations generally require the use of multiple antibodies specific to each chain of the CD8⁺ T-cell. For example, methods to determine the amounts of various CD8⁺ T cell subpopulations may require use of multiple anti-CD8α as well as anti-CD8αβ and anti-CD8β antibodies. While quantifiable, an efficient, cost effective method of testing and monitoring disease progression utilizing this data has yet to be described.

Accordingly, it would be beneficial to obtain more efficient, less expensive, improved diagnostic methods for the measurement of CD8⁺ T-cell subpopulations and evaluating the cellular impact of immunodeficiency viruses on these subpopulations.

### SUMMARY

The present disclosure describes a method of monitoring disease progression in a mammal positive for immunodeficiency virus. The method may include collecting blood cells from a mammal to obtain a first blood sample; adding antibodies to at least CD4 and CD8 to the first blood sample; scanning the first blood sample to produce a first multivariate dot plot; quantifying at least CD4⁺ and CD8⁺ blood cell populations using the first multivariate dot plot; calculating a ratio of the CD4⁺ to CD8⁺ blood cells to produce a first ratio of the first multivariate dot plot; quantifying a CD8αβ^{low} subpopulation using the first multivariate dot plot; calculating the percentage of the CD8αβ^{low} subpopulation of CD8⁺ blood cells to produce a second ratio of the first multivariate dot plot; calculating a ratio of the second ratio to the first ratio to produce a third ratio of the first multivariate dot plot; graphing the third ratio against the first ratio to produce a first point. The method may further include collecting a second blood cell sample from the mammal; adding antibodies to at least CD4 and CD8 to the second blood cell sample; scanning the second blood cell sample to produce a second multivariate dot plot; quantifying at least CD4⁺ and CD8⁺ blood cell populations using the second multivariate dot plot; calculating a ratio of the CD4⁺ to CD8⁺ to produce a first ratio of the second multivariate dot plot; quantifying the CD8αβ^{low} subpopulation of CD8⁺ blood cells using the second multivariate dot plot; calculating the percentage of the CD8αβ^{low} subpopulation of CD8⁺ blood cells to produce a second ratio of the second multivariate dot plot; calculating a ratio of the second ratio to the first ratio to produce a third ratio of the second multivariate dot plot; graphing the third ratio against the first ratio to produce a second point; comparing the first point to the second point to determine an extent of disease progression.

The disclosure also describes a method including obtaining a blood cell sample from at least one mammal; providing antibodies to at least two clusters of differentiation to the blood cell sample; scanning the blood cell sample to produce a multivariate dot plot; quantifying at least two blood cell populations based on their clusters of differentiation by using the multivariate; dot plot; calculating a ratio of the at least two blood cell populations to each other to produce a first ratio; quantifying at least one blood cell subpopulation of at least one of the at least two blood cell populations based on their cluster of differentiation by using the multivariate dot plot; calculating the percentage of the at least one blood cell subpopulation of the at least one of the at least two blood cell populations to produce a second ratio; calculating a ratio of the second ratio to the first ratio to produce a third ratio; and graphing the third ratio against the first ratio for the blood sample to identify cellular impact of an immunodeficiency virus on blood cells.

An additional method described in the disclosure may include obtaining a blood cell sample from a mammal; adding comprising antibodies to at least CD4 and CD8; scanning the blood cell sample to produce a multivariate dot plot; quantifying CD4⁺ and CD8⁺ blood cells using the multivariate dot plot; calculating a ratio of the CD4⁺ and CD8⁺ blood cells to produce a first ratio; quantifying a subpopulation of CD8⁺ blood cells using the multivariate dot plot; calculating the percentage of the subpopulation of CD8⁺ blood cells to produce a second ratio; calculating a ratio of the second ratio to the first ratio to produce a third ratio; utilizing the third ratio against the first ratio for each blood sample to identify cellular impact of an immunodeficiency virus on blood cells.

### BRIEF DESCRIPTION OF THE FIGURES

Various embodiments of the present disclosure will be described herein below with reference to the following figures wherein:

FIG. 1A depicts a fluorescent dot plot of an FIV⁺ blood sample;

FIG. 1B depicts a fluorescent dot plot of an FIV⁻ blood sample;

FIG. 2A depicts a fluorescent dot plot of an FIV⁺ blood sample;

FIG. 2B depicts another fluorescent dot plot of an FIV⁺ blood sample;

FIG. 2C depicts yet another fluorescent dot plot of an FIV⁺ blood sample;

FIG. 3A is a histogram of percent values for CD8αβ^{low} for a population of FIV⁺ cats;

FIG. 3B is a histogram of percent values for CD8αβ^{low} for a population of FIV⁻ cats along with a line representing the results of FIG. 3A;

FIG. 4A is a histogram of the CD4⁺/CD8⁺ ratio for the FIV⁻ samples including those of FIG. 3B;

FIG. 4B is a histogram of the CD4⁺/CD8⁺ ratio for the FIV⁺ samples of FIG. 3A; and

FIG. 5 is a bivariate plot of CD4⁺/CD8⁺ ratio verses % CD8αβ^{low}/(CD4⁺/CD8⁺) for both FIV⁺ and FIV⁻ cats.

### DETAILED DESCRIPTION

The present disclosure provides a simple, accurate method for assessing the impact of an immunodeficiency virus on the blood cells of mammals afflicted with immunodeficiency viruses. The disclosure also provides a method to obtain better resolution between immunodeficiency virus negative and immunodeficiency virus positive populations. The disclosure further provides a method of differentiating between FIV infected cats and cats vaccinated against FIV. The methods described are independent of total lymphocyte count and any variation thereof.

Assessing the impact of an immunodeficiency virus on blood cells of mammals in accordance with the present disclosure may be achieved by obtaining blood cells from both healthy mammals and those afflicted with immunodeficiency virus. Antibodies to least two clusters of differentiation may be added to the blood cell samples and each sample may be scanned to produce a multivariate dot plot. The multivariate dot plot may be used to quantify the clusters of differentiation. The ratio of the clusters of differentiation may provide a first ratio. A subpopulation of at least one of the clusters of differentiation may also be quantified using the multivariate dot plot. The percentage of the subpopulation may provide a second ratio. A third ratio may be produced by calculating the ratio of the second ratio to the first ratio. The third ratio may be plotted against the first ratio to produce a point on a graph for identifying the cellular impact of an immunodeficiency virus. In embodiments, samples from immunodeficiency virus positive and immunodeficiency virus negative mammals be taken and each sample may provide a point on the graph and these points may result in a diagnostic curve. In embodiments, the curve may be used to determine whether cells from a particular mammal are affected by immunodeficiency virus. The location of the point derived by graphing the third ratio against the second ratio may also be used to evaluate any changes in a particular mammal's cells following treatment or during disease progression.

As described above, the CD8⁺ T-cells include several subpopulations, The present disclosure provides for the application of a single CD8⁺ antibody for detecting total CD8⁺ as well its sub-populations. This method does not require the use of multiple sub-type antibodies but, instead, provides for the labeling of whole blood cells using one CD8⁺ antibody. Antibodies that may be used for additional T-cell markers include, for example, antibodies such as CD4, for example, 3-4F4 (Southern Biotech), RFT-4g (Southern Biotech), VPG34 (Serotec AbD) and RPA-T4 (Serotec AbD); CD5, such as f43 (Southern Biotech), UCHT2 (Southern Biotech), FE1.1B11 (Serotec AbD) and MF7-14.5 (Serotec AbD); and CD8, such as fCD8 (Southern Biotech), UCH-T4 (Southern Biotech), LT8 (Serotec AbD) and VPG9 (Serotec AbD). An antibody that may be used for a B-cell marker is CD21, such as CA2.1D6 (Serotec AbD), LB21 (Serotec AbD), and BU32 (Southern Biotech). Another antibody used for a monocyte marker is CD14, such as TüK4 (Serotec AbD) and UCHM-1 (Southern Biotech). The isotype control antibody for these antibodies may be IgGs with three-color fluorescence such as TC012 (Serotec AbD). The blood is then scanned to produce a multivariate dot plot representing each cell by corresponding fluorescence signal levels from each specific antibody labels on the cell.

The methods of the present disclosure also include a method for quantifying CD8⁺ subpopulations. The multivariate dot plot may be used to determine the amount of each CD8⁺ subpopulation as a percentage of the total CD8⁺ population. The methods of the present disclosure involve separation and quantification of these subpopulations as a diagnostic tool for immunodeficiency viruses.

Identifying the cellular impact of an immunodeficiency virus in a mammal may also be accomplished using the method of the disclosure. In order to identify whether the mammal is exhibiting active immunodeficiency virus, a blood sample may be taken from the mammal. A CD8⁺ antibody may then be added to the blood sample, the red blood cells (RBCs) may be lysed, and the sample may be scanned. A multivariate dot plot may be produced by the scan. The percentage of CD8αβ^{low} cells as a percentage of the total CD8⁺ population may be used to evaluate immunodeficiency virus activity and/or the mammal's physiological response to the virus.

Determining disease progression in a mammal positive for immunodeficiency virus may also be achieved using the methods of the present disclosure. The method may involve collecting a first blood sample from a mammal, adding one CD8⁺ antibody, adding at least one additional antibody selected from the group consisting of CD4, CD5, CD14, CD21, and CD61 to the first blood sample to form a first blood-antibody mixture, and scanning the blood-antibody mixture to produce a first multivariate dot plot. Next the method includes collecting a second blood sample from the mammal at a subsequent point in time, adding one CD8 antibody, adding at least one additional antibody selected from the group consisting of CD4, CD5, CD14, CD21, and CD61 to the second blood sample to form a second blood-antibody mixture, scanning the second blood-antibody mixture to produce a second multivariate dot plot, and comparing the first multivariate dot plot to the second multivariate dot plot to determine the extent of disease progression. By comparing the multivariate dot plots, cellular impact of an immunodeficiency virus may be monitored.

Another diagnostic measure used in immunodeficiency virus monitoring is the CD4⁺:CD8⁺ ratio. In accordance with the present disclosure, the ratio of the percentage of CD8αβ^{low} in the total CD8⁺ population to the CD4⁺:CD8⁺ ratio may be plotted against the CD4⁺:CD8⁺ ratio. Mammals infected with an immunodeficiency virus may have a lower ratio as compared to uninfected mammals.

The term “mammals” includes, for example, mice, cats, simians, humans, and the like, as are commonly known to be mammals.

As stated above, in order to perform the method of the disclosure, blood may be drawn. The blood may then be mixed with at least a reagent specific to CD8⁺ T-cells. Reagents that may be used to label CD8⁺ T-cells include, for example, mouse anti-mammal CD8 mouse anti-mammal CD8αβ, mouse anti-mammal CD8β, mouse anti-mammal CD8α and combinations thereof, and the like. Antibodies for labeling additional blood and/or leukocyte components for fluorescent labeling which may be added include those with the purview of those skilled in the art. A lysing agent may be added to eliminate the RBCs. The labeled blood may then be scanned on a flow cytometer or other device for scanning blood cells. In embodiments, a lysing agent may be added prior to scanning the blood cells in the flow cytometer. In embodiments, the blood cells may be centrifuged prior to scanning in the flow cytometer.

Although described with regard to a fluorescent dot plot produced by a flow cytometer, any method capable of detecting antibodies, with or without the use of fluorescence is contemplated by this disclosure. The flow cytometer produces a fluorescent dot plot based upon the binding of the labeled antibody. The population of CD8αβ^{low} reflected in the florescent dot plot may then be evaluated. in healthy cats the percentage of CD8⁺ T-cells of the CD8αβ^{low} subtype is relatively low, typically less than 5%. By contrast, in cats having acute-stage FIV infections, the percentage of CD8³⁰ T-cells are of the CD8αβ^{low} subtype is higher, typically 20% or more.

The CD4⁺:CD8⁺ ratio and the percentage of CD8αβ^{low} used individually may not provide a complete diagnostic picture. However, quantification of the ratio of the percentage of CD8αβ^{low} cells (in the total CD8⁺ population) to the CD4⁺:CD8⁺ ratio, (hereinafter “% CD8αβ^{low}/(CD4⁺:CD8⁺)”) may be used to provide improved diagnostic information. Specifically, by utilizing a ratio to ratio measurement, uncertainties caused by the use of only absolute counts which are derived as a percent of total lymphocyte counts is reduced. Additionally, in cats having the same CD4⁺:CD8⁺ ratio, FIV⁺cats may also exhibit a higher % CD8αβ^{low}/(CD4³⁰ :CD8³⁰) ratio. The curve of the graph of % CD8αβ^{low}/(CD4⁺:CD8⁺) ratio provides a trend for both FIV⁺ and FIV⁻ cats. In embodiments, the % CD8αβ^{low}/(CD4⁺:CD8⁺) may be graphically plotted against the CD4⁺:CD8⁺ to provide a numeric evaluation of the impact of the virus on CD8⁺ T-cells.

Monitoring of immunodeficiency virus progression in a mammal may include obtaining a blood sample from the infected mammal at intervals of, for example, from about 1 week to about 6 months, in embodiments, about 1 month to about 3 months. Depending on the rate of progression of the illness, the rate of obtaining the samples may vary. After obtaining a sample, antibodies specific to at least one sub-population of CD8⁺ T-cells may be added to the sample. The sample may then he scanned in a flow cytometer. Evaluation of disease progression may involve, for example, comparing the percentage of CD8αβ^{low} T-cells in a current scan, to the prior scan(s). In embodiments, the method of monitoring immunodeficiency virus progression may involve, for example, comparing the % CD8αβ^{low}/(CD4⁺:CD8⁺) ratio of the first sample to that of the second sample. The ratio of the % CD8αβ^{low} /(CD4⁺:CD8³⁰) may be graphed against the (CD4⁺:CD8⁺) ratio. An increase in the % CD8αβ^{low} over time may indicate increased immune response to the virus. A decrease in the % CD8αβ^{low} may indicate that immune response to the virus is decreasing.

The following Examples are being submitted to illustrate embodiments of the present disclosure. These Examples are intended to be illustrative only and are not intended to limit the scope of the present disclosure. Also, parts and percentages are by weight unless otherwise indicated, As used herein, “room temperature” refers to a temperature of from about 20° C. to about 30° C.

### EXAMPLES

### Example 1

Blood samples of FIV⁺ and FIV⁻ cats were collected in an ethylenediaminetetraacetic acid (EDTA) tube from the local clinics and shelters. A 100 μl aliquot of each blood sample was incubated with 5 μl mouse anti cat CD4:fluoroseein isothiocyanate (FITC) (AbD Serotec), 5 μl mouse anti cat CD8αβ:R-phycoerythrin (RPE) (AbD Serotec) and CD61:Alexa Fluor 647 fluorescent dye for 30 minutes at room temperature and kept in dark unless stated otherwise.

To process the samples for lymphocyte analysis, 2 ml of 1+ lysing solution diluted with distilled water (BD PharmLyse) was added to 0.1 ml EDTA blood plus antibody mixture in a sterile 12×75 mm (BD Biosciences). The sample was gently vortexed immediately and incubated at room temperature for 15 minutes. Then the tube was centrifuged in a Horizon Premier centrifuge (The Drucker Co.) at 200 g-force (approximately 1100 rpm) for 5 minutes and the resulting supernatant was aspirated, leaving a cell pellet in the tube.

The cell pellet was washed with 2 ml 1+ phosphate buffered saline (PBS) (Mediatech Inc) containing 1% fetal bovine sera (FBS) (SAFC Biosciences). The washed cell pellet was then centrifuged at 200 g-force for 5 minutes and the supernatant was carefully aspirated. The cell pellet was resuspended in 0.4 ml 1+ PBS containing 1% PBS. This sample was then run on a flow cytometer Accuri C6 (Accuri).

Cluster of differentiation 61 positive (CD61³⁰ ) platelets labeled with an antibody (VI-PL2, BD) were measured in one of the four photomultiplier tubes of the instrument. The lymphocytes were identified based on their light scatter signals and then further sub-classified as CD4⁺ and CD8⁺ positive cells by measuring the FITC and RPE fluorescence in two other photomultipliers. A total of about 10,000 lymphocyte events were acquired for each sample and analyzed by the CFlow software (Accuri). CFlow software was used for data analysis.

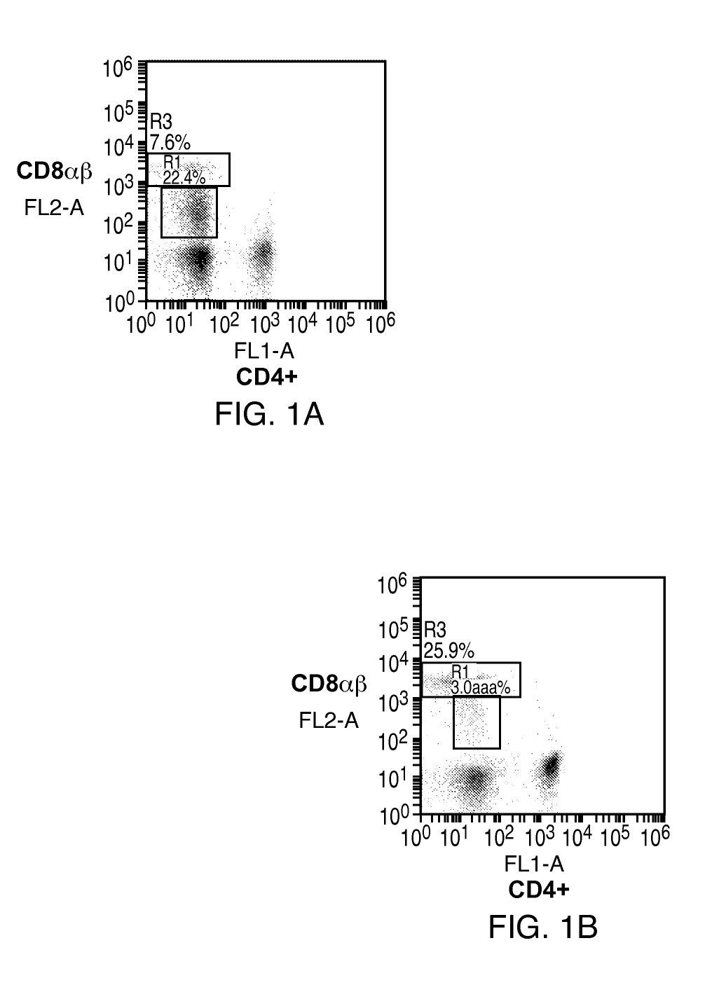

Referring in detail to the Figures in which like reference numerals are applied to like elements in the various views, FIGS. 1A and 1B depict flow cytometery fluorescence dot plots resulting from two different feline blood samples. FIG. 1A is a scan of blood from an FIV⁺ cat while FIG. 1B is a scan of blood from an FIV⁺ cat. CD4⁺ cells are shown in the lower right hand quadrant of the graph. The CD8⁺ labeled cells had two populations CD8αβ^{low} and CD8αβ^{high}. The CD8αβ^{low} was greater in the FIV⁺ cat (FIG. 1A) than in the FIV⁻ cat (FIG. 1B).

### Example 2

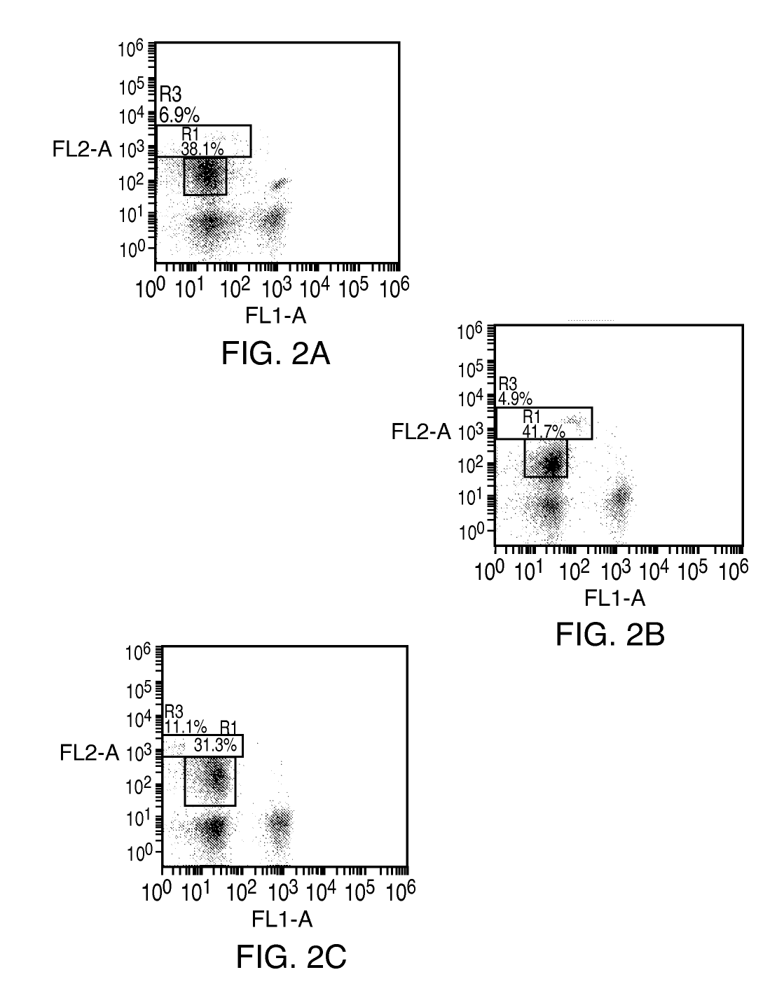

Blood samples of three different FIV⁺ cats were collected in separate EDTA tubes. Each of these cats had relatively bad gingivitis, which is often an indication of advanced immune deficiency in PTV infected cats. FIGS. 2A-C depict fluorescence dot plots for CD4⁺ and CD8⁺ labeled lymphocytes for each FIV⁺ cats. The CD8αβ^{low} sub-population was relatively high in each of the FIV⁺ cats.

### Example 3

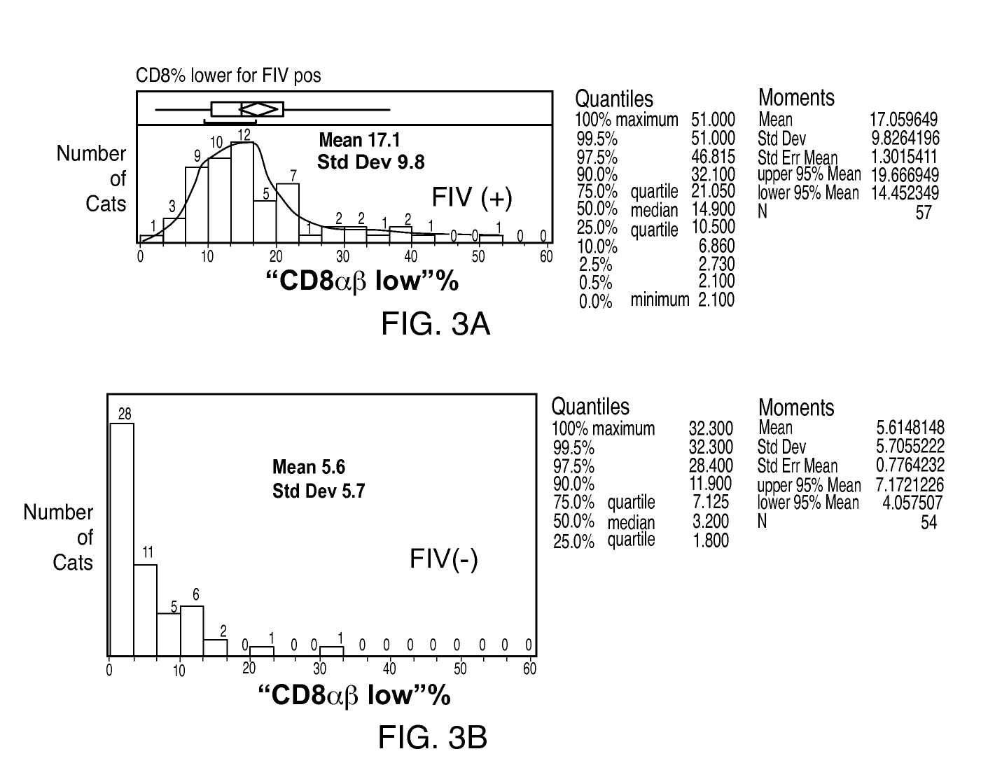

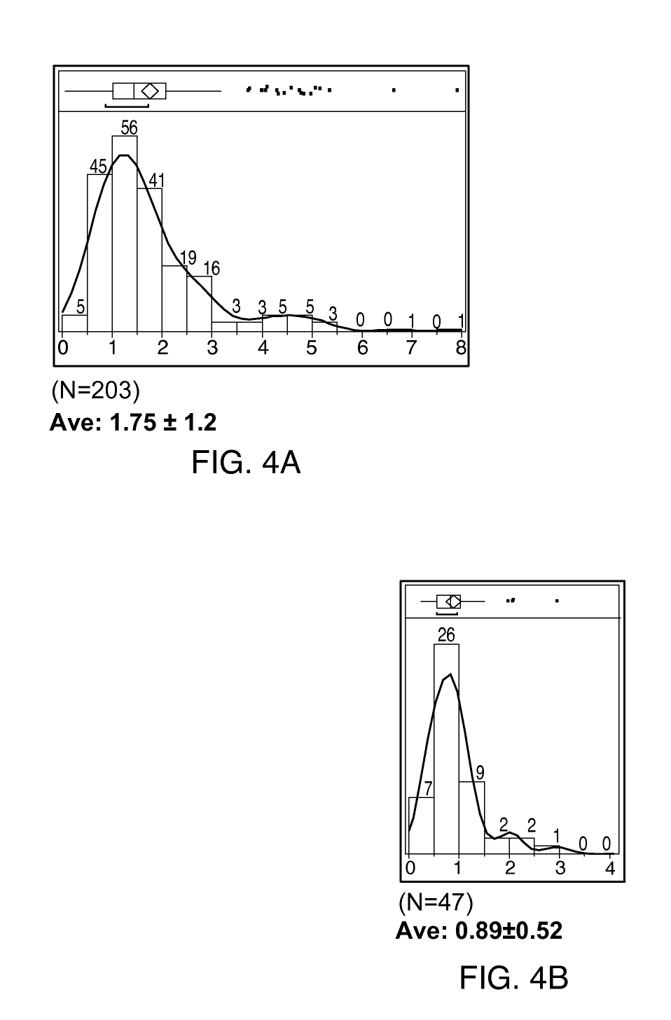

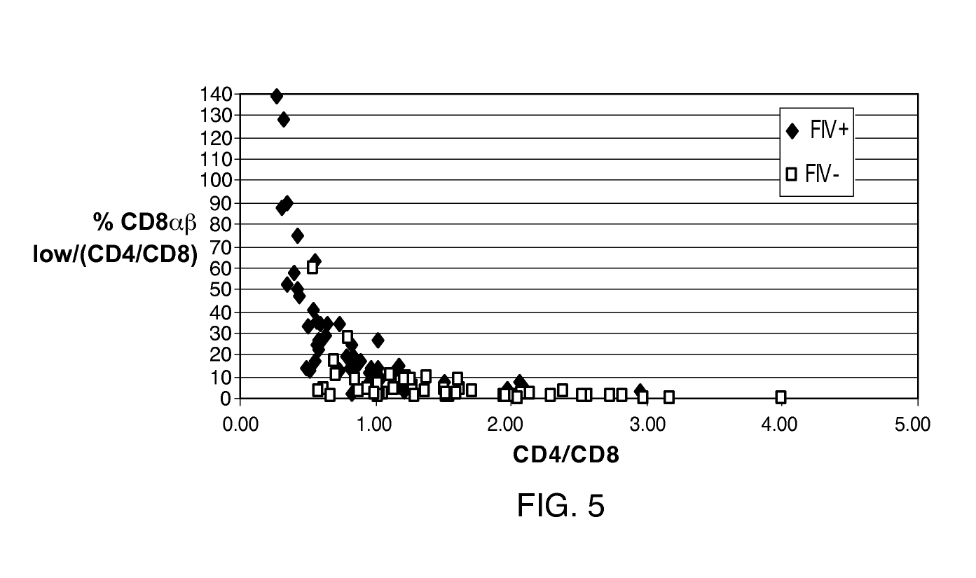

The percentage of CD8αβ^{low} cells as compared to the total number of cells the sample in 57 FIV⁺ and 54 FIV⁻ feline blood samples are shown FIGS. 3A and 3B. FIGS. 4A and 4B show the CD4⁺/CD8⁺ ratio 203 FIV⁻ cats and 47 FIV⁺ cats, which include those of FIGS. 3A and 3B. The ratio of the percentage of CD8αβ^{low} in the CD8⁺ population to CD4⁺/CD8⁺ relative to the CD4⁺/CD8⁺ ratio is charted in FIG. 5. Each of the samples was collected, prepared, and measured the same way as described in Example 1. Statistical analysis was performed using a commercial JMP software package. Consistent with the graphical demonstration in FIGS. 1 and 2 for individual cats, FIG. 5 shows that, in general the ratio of CD8αβ^{low} to CD4⁺/CD8⁺ was higher for the FIV⁺ population (solid diamonds) compared to the FIV⁻population (open squares).

Although various specific embodiments of the present invention have been described herein, it is to be understood that the invention is not limited to those precise embodiments and that various changes or modifications can be affected therein by one skilled in the art without departing from the scope and spirit of the invention. In addition, while the specific examples primarily relate to Feline Immunodeficiency Virus (FIV), one skilled in the art would understand that the techniques and methods taught herein have utility in the diagnosis of Human Immunodeficieney Virus (HIV).

What is claimed is:

1. A method of monitoring disease progression in a mammal positive for immunodeficiency virus comprising:
collecting blood cells from a mammal to obtain a first blood sample;
adding antibodies to at least CD4 and CD8 to the first blood sample;
scanning the first blood sample to produce a first multivariate dot plot;
quantifying at least CD4⁺ and CD8⁺ blood cell populations using the first multivariate dot plot;
calculating a ratio of the CD4³⁰ to CD8⁺ blood cells to produce a first ratio of the first multivariate dot plot;
quantifying a CD8αβ^{low} subpopulation using the first multivariate dot plot;
calculating the percentage of the CD8αβ^{low} subpopulation of CD8⁺ blood cells to produce a second ratio of the first multivariate dot plot;
calculating a ratio of the second ratio to the first ratio to produce a third ratio of the first multivariate dot plot;
graphing the third ratio against the first ratio to produce a first point;
collecting a second blood cell sample from the mammal;
adding antibodies to at least CD4 and CD8 to the second blood cell sample;
scanning the second blood cell sample to produce a second multivariate do plot;
quantifying at least CD4⁺ and CD8⁺ blood cell populations using the second multivarlate dot plot;
calculating a ratio of the CD4⁺ to CD8⁺ to produce a first ratio of the second multivariate dot plot;
quantifying the CD8αβ^{low} subpopulation of CD8⁺ blood cells using the second multivariate dot plot;
calculating the percentage of the CD8αβ⁺ subpopulation of CD8⁺ blood cells to produce a second ratio of the second multivariate dot plot;
calculating a ratio of the second ratio to the first ratio to produce a third ratio of the second multivariate dot plot;
graphing the third ratio against the first ratio to produce a second point;
comparing the first point to the second point to determine an extent of disease progression.

2. The method of claim 1, further comprising adding a lysing agent to the first blood cell sample prior to scanning.

3. The method of claim 1, wherein the mammal is selected from the group consisting of mouse, cat, simian, and human.

4. The method of claim 1, wherein the antibodies to the at least CD4 and CD8 further comprise a fluorescing agent.

5. The method of claim 1, wherein the immunodeficiency virus is selected from the group consisting of MIV, FIV, SIV, and HIV.

6. A method comprising:
obtaining a blood cell sample from at least one mammal;
providing antibodies to at least two clusters of differentiation to the blood cell sample;
scanning the blood cell sample to produce a multivariate dot plot;
quantifying at least two blood cell populations based on their clusters of differentiation by using the multivariate dot plot;
calculating a ratio of the at least two blood cell populations to each other to produce a first ratio;
quantifying at least one blood cell subpopulation of at least one of the at least two blood cell populations based on their cluster of differentiation by using the multivariate dot plot;
calculating the percentage of the at least one blood cell subpopulation of the at least one of the at least two blood cell populations to produce a second ratio;
calculating a ratio of the second ratio to the first ratio to produce a third ratio; and
graphing the third ratio against the first ratio for the blood sample to identify cellular impact of an immunodeficiency virus on blood cells.

7. The method of claim 6, wherein the antibodies are antibodies to clusters of differentiation selected from the group consisting of CD4, CD5, CD14, CD21, CD61, CD8 and combinations thereof.

8. The method of claim 7, wherein the antibody comprises an antibody to a subpopulation of CD8 selected from the group consisting of CD8αβ^{low}, CD8αβ^{high}, CD8α, CD8β and combinations thereof.

9. The method of claim 6, wherein the antibodies to at least two chaster of differentiation comprise a CD4 antibody and a CD8 antibody.

10. The method of claim 6, wherein the at least one blood cell subpopulation comprises CD8αβ^{low}.

11. The method of claim 6, wherein the blood cell sample further comprises a lysing agent.

12. The method of claim 6, wherein the antibodies to the at least two cluster of differentiation further comprise a fluorescing agent.

13. The method of claim 6, wherein the mammal is selected from the group consisting of mouse, cat, simian, and human.

14. The method of claim 6, wherein the mammals are cats.

15. The method of claim 6, wherein the immunodeficiency virus is selected from the group consisting of MIV, FIV, SIV, and HIV.

16. The method of claim 6, wherein the scanning is conducted on a flow cytometer.

17. A method comprising:
obtaining a blood cell sample from a mammal;
adding comprising antibodies to at least CD4 and CD8;
scanning the blood cell sample to produce a multivariate dot plot;
quantifying CD4⁺ and CD8³⁰ blood cells using the multivariate dot plot;
calculating a ratio of the CD4³⁰ and CD8⁺ blood cells to produce a first ratio;
quantifying a subpopulation of CD8⁺ blood cells using the multivariate dot plot;
calculating the percentage of the subpopuiation of CD8⁺ blood cells to produce a second ratio;
calculating a ratio of the second ratio to the first ratio to produce a third ratio;
utilizing the third ratio against the first ratio for each blood sample to identify cellular impact of an immunodeficiency virus on blood cells.

18. The method of claim 17, wherein the subpopulation of CD8⁺ blood cells is selected from the group consisting of CD8αβ^{low}, CD8αβ^{high}, CD8α, CD8β and combinations thereof.

19. The method of claim 17, further comprising adding a lysing agent to the blood sample.

20. The method of claim 17, wherein the subpopulation of CD8⁺ blood cells comprises CD8αβ^{low}.
